(19) United States
(12) Patent Application Publication
(10) Pub. No.: US 2009/0124911 A1
(43) Pub. Date: May 14, 2009

(54) Sphygmomanometer capable of adjusting a viewing angle of a display screen

(75) Inventors: Chia Chien Lin, Taipei County (TW); Chun-Hung Chu, Taipei County (TW); Su-Chen Cheng, Taipei (TW)

Correspondence Address: BACON & THOMAS, PLLC, 625 SLATERS LANE, FOURTH FLOOR, ALEXANDRIA, VA, 22314-1176, US

(73) Assignee: HEALTH & LIFE CO., LTD., CHUNG HO CITY (TW)

(21) Appl. No.: 12/007,501

(22) Filed: Jan. 11, 2008

(30) Foreign Application Priority Data
Nov. 12, 2007 (TW) 096219062

(51) Int. Cl. A61B 5/022

(52) U.S. Cl. 600/490

(57) ABSTRACT

A sphygmomanometer capable of adjusting a viewing angle of a display screen includes a housing, and the housing includes a lower casing and an upper casing. The lower casing includes a pivotal axle and a position limit mechanism. The upper casing has a containing space for containing a rotating body, and a display screen installed on a side of the rotating body, and a shaft protruded from another side of the rotating body which faces the lower casing. The shaft is sheathed onto the pivotal axle for the use of the sphygmomanometer. When the sphygmomanometer is worn onto a user's wrist or arm, the pivotal axle and the shaft are operated to turn the display screen on the rotating body to an appropriate position, so as to facilitate the user or medical professionals to view the reading of the measurement on the display screen.

### BACKGROUND OF THE INVENTION

1. Field of the Invention

The present invention relates to a sphygmomanometer capable of adjusting a viewing angle of a display screen, and more particularly to a sphygmomanometer that allows users to adjust a screen display to the best viewing angle to facilitate measuring the blood pressure by themselves or medical professionals.

2. Description of the Related Art

Heart is one of the important organs in a human body, and cardiovascular disease becomes one of the major fatal causes of modern people. Physiological information such as blood pressure measurements and irregular pulsations (or arrhythmias) are very important to early discoveries and treatments of the disease and regular tracing and examination of physical conditions.

Referring to FIG. 1, the present display screen of a general electronic sphygmomanometer for displaying blood pressure is of a fixed type, particularly for a wrist sphygmomanometer that is worn at a user's wrist. The wrist sphygmomanometer usually comes with a small size for an easy carry, and users need not to take of the cloth or roll up the sleeve for the measurement of blood pressure, and thus this type of sphygmomanometer becomes increasingly popular. When a user measures blood pressure, a main body of the sphygmomanometer is placed at the middle of an internal side of the user's wrist. After the user has measured and checked the measurements, the user usually needs to turn the wrist outward to an angle to obtain the best viewing angle of the display screen, since the display screen is situated perpendicularly on the internal side of the user's wrist. Further, if a medical professional needs to view the measurements on the screen display, the wrist has to be turned to a larger angle, and thus causing discomfort to users, particularly to the elderly users.

### SUMMARY OF THE INVENTION

In view of the foregoing shortcomings of the prior art that a user cannot obtain a good viewing angle of the reading of the processed blood pressure on a display screen of a traditional sphygmomanometer, the inventor of the present invention based on years of experience in the related industry to conduct extensive experiments and researches, and finally developed a sphygmomanometer capable of adjusting a viewing angle of a display screen to overcome the shortcomings of the prior art.

It is a primary objective of the present invention to overcome the foregoing shortcomings of the prior art by providing a sphygmomanometer that can turn the angle of a display screen to facilitate users or medical professionals to view the reading of a measurement without moving the sphygmomanometer and changing the user's posture.

To achieve the foregoing objective, the present invention provides a sphygmomanometer including a housing, and the housing includes a lower casing with a pivotal axle, an upper casing having a shape corresponding to the lower casing, a hollow containing space disposed inside the upper casing, a rotating body disposed in the containing space, a shaft protruded from the rotating body which faces a side of the lower casing and precisely sheathed onto the pivotal axle, and a display screen installed onto the rotating body for displaying a reading of blood pressure measured by a user. With the operation of the shaft and the pivotal axle, the display screen can be turned to a position and a viewing angle to facilitate the user or a medical professional to view the reading of the measurement.

Further, the rotating body includes a positioning element, a first positioning point disposed at a front end of the positioning element, a plurality of second positioning points disposed on the lower casing and corresponding to the first positioning point, so that the rotating body can be moved on the lower casing, and provides the positioning function and prevent the sphygmomanometer from shifting or shaking easily.

To make it easier for the examiner to understand the object, shape, structure, device, characteristics and functions of this invention, the specification accompanied by the drawings is described as follows.

### BRIEF DESCRIPTION OF THE DRAWINGS

FIG. 1 is a schematic view of operating a prior art sphygmomanometer;

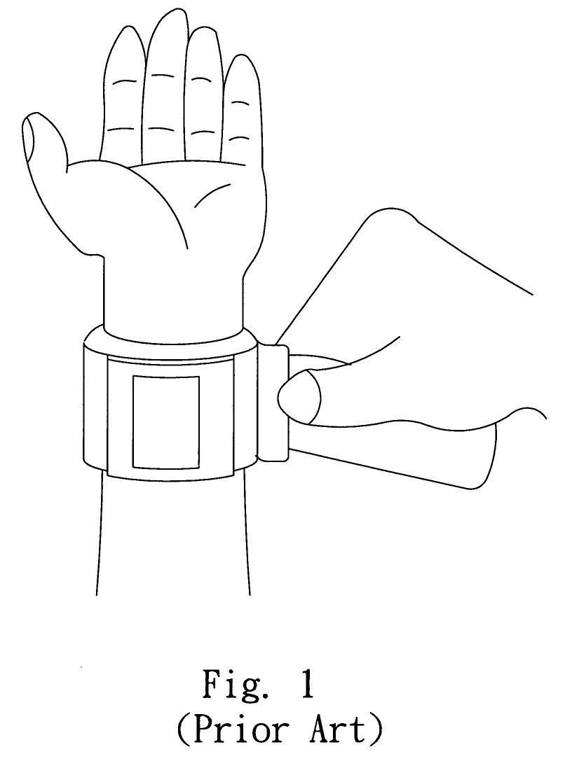

FIG. 2 is a perspective view of the present invention;

FIG. 3 is an exploded view of a housing in accordance with the present invention;

FIG. 4 is an exploded view of a portion of a housing in accordance with the present invention viewing from another angle;

FIG. 5 is a schematic view of a rotating body connected to a position limit mechanism in accordance with the present invention;

FIG. 6 is a schematic view of operations of a first positioning point and a second positioning point in accordance with the present invention;

FIG. 7 is a section view of a housing of the present invention; and

FIG. 8 is a schematic view of operating a sphygmomanometer of the present invention.

### DETAILED DESCRIPTION OF THE PREFERRED EMBODIMENTS

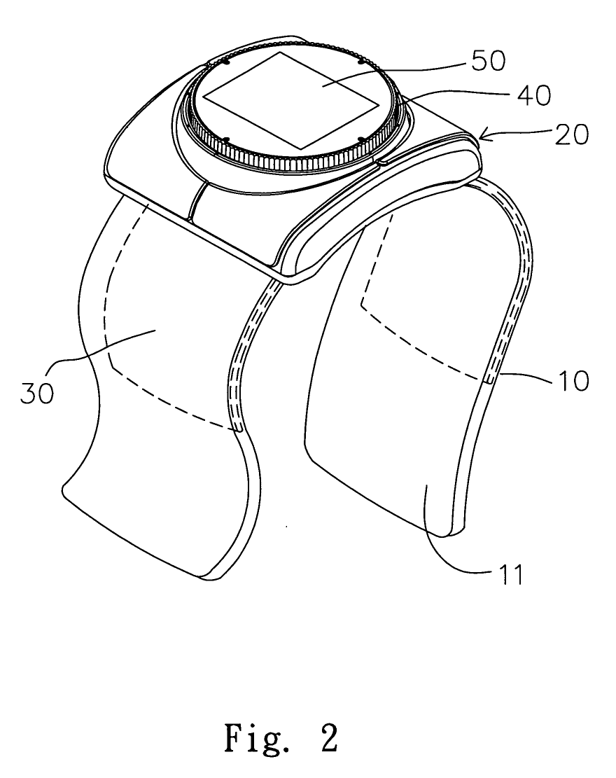

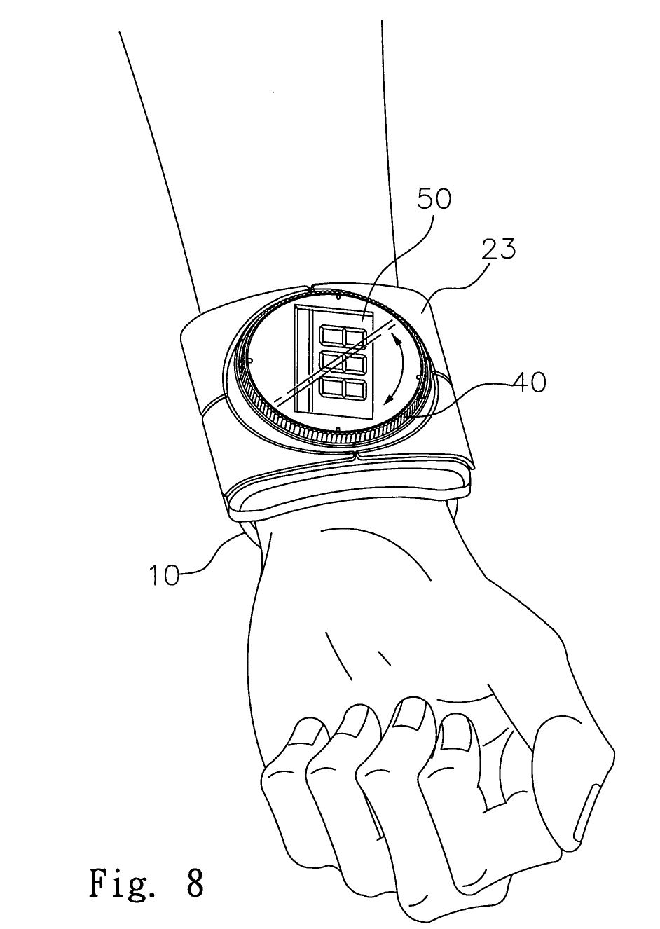

Referring to FIG. 2 for a sphygmomanometer capable of adjusting a viewing angle of a display screen in accordance with the present invention, this preferred embodiment is a wrist sphygmomanometer. The sphygmomanometer includes a cuff 10, an adhesive member 11 disposed separately on both ends of the cuff 10 for fixing the cuff 10 around a user's wrist (as shown in FIG. 8), a housing 20 disposed on a surface of the cuff 10, and an air puff 30 installed in the cuff 10 for inflating or deflating air to carry out the measurement.

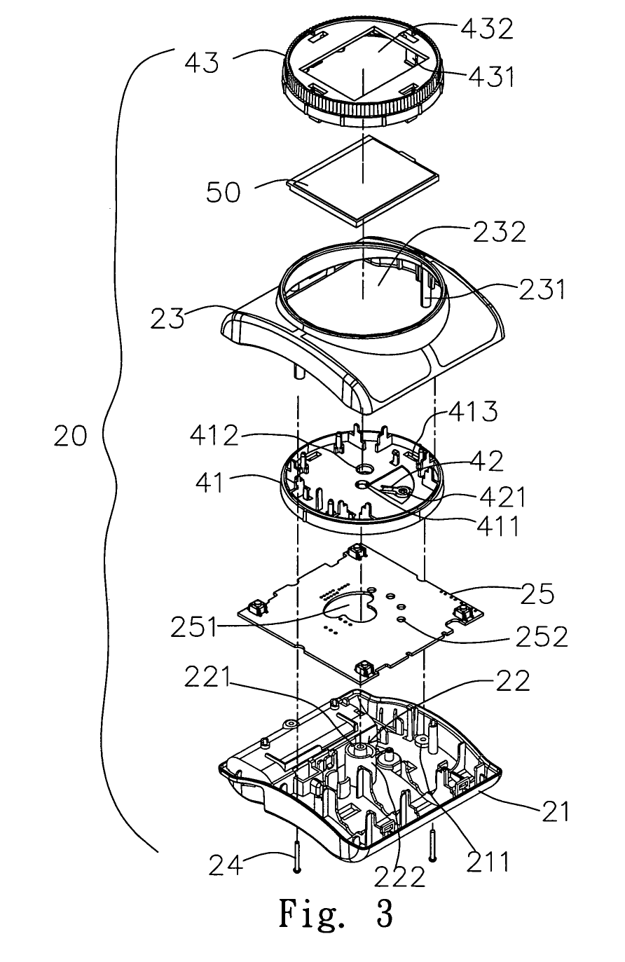

In FIG. 3, the housing 20 includes a lower casing 21, which is a rectangular housing in this embodiment, and the lower casing 21 includes at least one screw hole 211 disposed around the periphery of the lower casing 2, a pivotal axle 22 protruded from the lower casing 2, a protruding pillar 221 disposed in the pivotal axle 22, and a bushing 222 sheathed around the periphery of the protruding pillar 221, wherein a predetermined interval is maintained between the bushing 222 and the protruding pillar 221.

Further, the lower casing 21 comes with a shape corresponding to the upper casing 23, and the upper casing 23 is a rectangular hollow casing in this embodiment, and the upper casing 23 includes a screw pillar 231 disposed around the upper casing 23 and corresponding to the screw hole 211 for passing a screw 24 into the screw hole 211, and securing the screw 24 to the screw pillar 231, such that the lower casing 21 and the upper casing 23 are combined to form the housing 20.

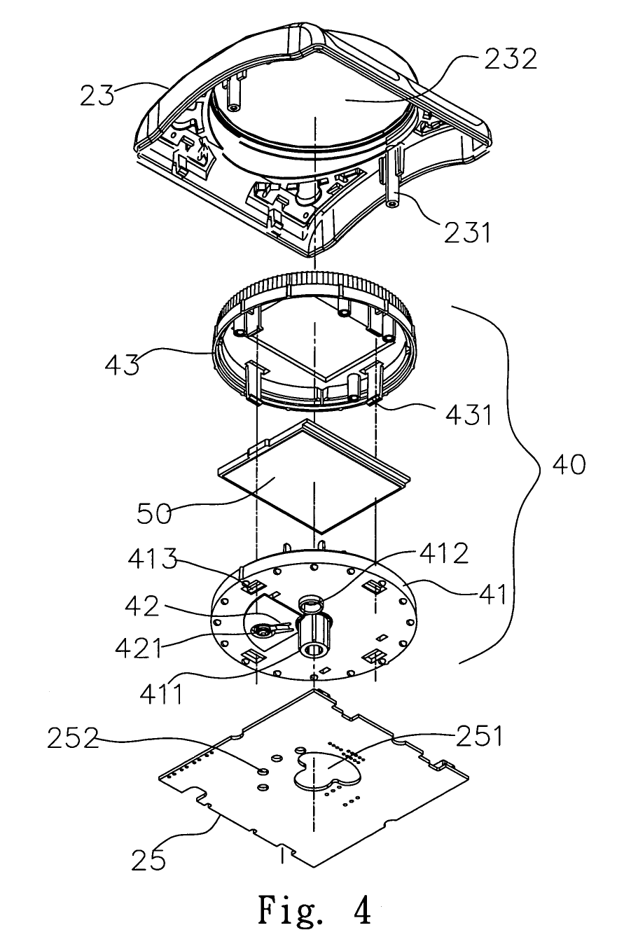

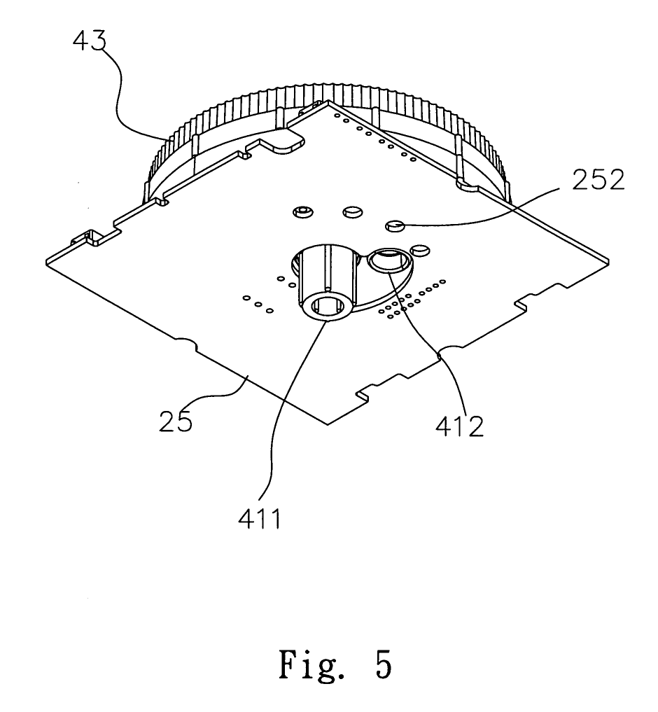

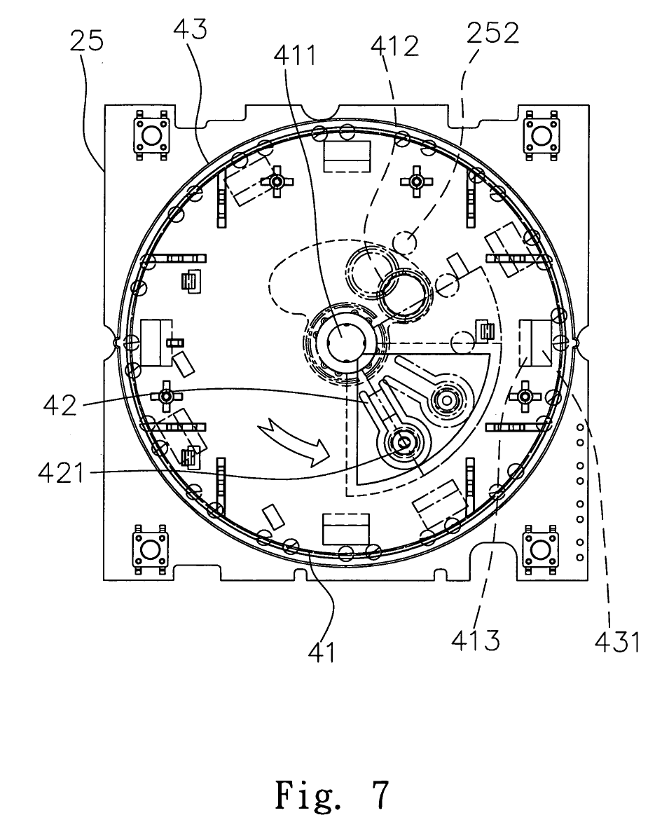

In FIGS. 3 and 4, the upper casing 23 includes a hollow containing space 232 which is a circular opening in this embodiment, and the containing space 232 accommodates a rotating body 40, and the rotating body 40 has a chassis 41, and the chassis 41 has a shaft 411 and a position limit pillar 412 protruded from the chassis 41 which faces a side of the lower casing 21, and the shaft 411 is a hollow tube in this embodiment, and the position limit pillar 412 is a protruding pillar shorter than the shaft 411. Further, a position limit mechanism 25 is installed between the lower casing 21 and the chassis 41, which is a standalone sheet of the lower casing. However, the persons skilled in the art still can integrally couple the position limit mechanism 25 with the lower casing. A position limit groove 251 is disposed on the position limit mechanism 25 and corresponding to the shaft 411 and the position limit pillar 412, and the position limit groove 251 is a cambered surface in this preferred embodiment, and the arc central position of the position limit groove 251 is provided for passing and connecting the shaft 411 between the bushing 222 and the protruding pillar 221, such that the shaft 411 can be rotated therein (as shown in FIG. 5), and the position limit pillar 412 is sheathed precisely onto the cambered surface of the position limit groove 251 and moved within the range of the cambered surface. Further, the chassis 41 includes a positioning element 42, which is an elastic sheet in this embodiment, and a first positioning point 421 is protruded from the front end of the positioning element 42 and corresponding to a plurality of second positioning points 252 disposed on the position limit mechanism 25, and the first positioning point 421 can be moved on the second positioning point 252, so as to achieve the positioning effect (as shown in FIG. 7).

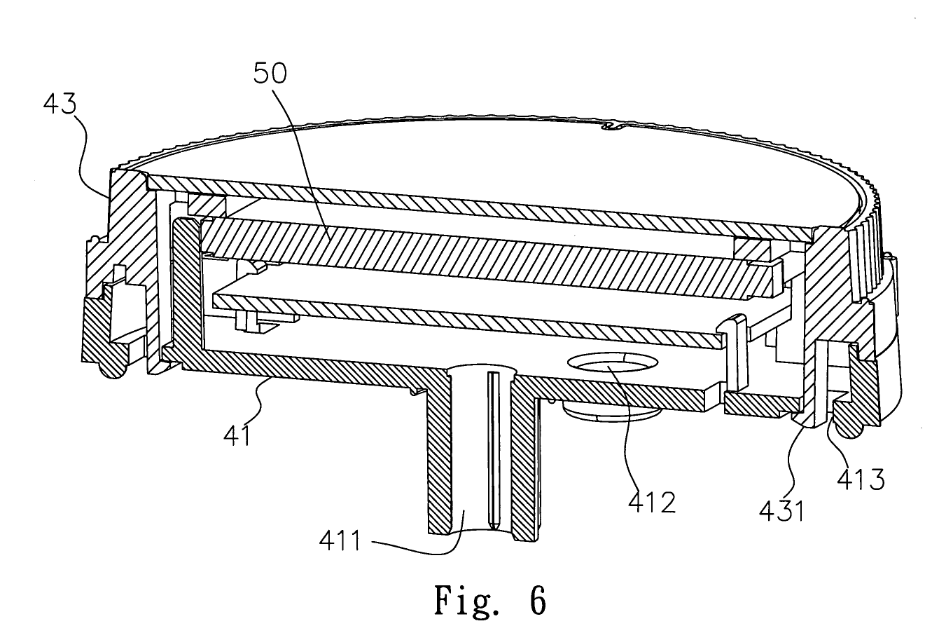

In FIGS. 3, 6 and 7, the chassis 41 has a hood 43 with a shape corresponding to the chassis 41, and the hood 43 has at least one embedding hook 431 disposed at a specific position. Further, an embedding groove 413 disposed at a periphery of the chassis 41 and corresponding to the embedding hook 431, such that the embedding hook 431 can be embedded into the embedding groove 413 to fix the hood 43 and the chassis 41 together (as shown in FIG. 6), and the hood 43 has an opening 432 for accommodating a display screen 50, and the display screen 50 is clamped precisely between the chassis 41 and the hood 43 (as shown in FIG. 4), and the display screen is electrically connected to a circuit control device in the housing 10 through the chassis 41 by an electric circuit (not shown in the figure), and the circuit control device and the position limit mechanism are integrated as a whole in this embodiment.

In FIGS. 2 and 8, the cuff 10 is wrapped to fix the housing 20 onto a user's wrist, and an air puff 30 in the cuff 10 is pressurized to a predetermined air pressure and then the pressurization is stopped, and the air in the air puff 30 is discharged stably to the outside, and the housing 20 can detect the pulses at systolic and diastolic blood pressures while the air is discharged slowly. The data of detected systolic and diastolic blood pressures are processed, and the processed result is sent to and displayed on the display screen 50. In the meantime, the rotating body 40 can be rotated at the housing 20 with the operation of the shaft 411 and the pivotal axle 22, so that the display screen 50 can be turned to a position and an angle to facilitate users to view and read the reading displayed on the display screen 50 (as shown in FIG. 8). When the shaft 411 is rotated, the limitation provided by the position limit pillar 412 and the position limit groove 251 restricts the angle of turning the display screen 50 in an acceptable range. With the first positioning point 421 and the second positioning point 252, the rotating body 40 can be turned to a specific position without a shift or shaking, so as to fix the rotating body 40 to a desired position.

In summation of the above description, the sphygmomanometer capable of adjusting a viewing angle of a display screen in accordance with the present invention comes with a simple structure, and achieves the effects of saving cost and enhancing the performance than the conventional structure and further complies with the patent application requirements and is submitted to the Patent and Trademark Office for review and granting of the commensurate patent rights.

What is claimed is:

1. A sphygmomanometer capable of adjusting a viewing angle of a display screen, including a housing, and said housing comprising:
a lower casing, having a pivotal axle therein;
an upper casing, disposed at the top of said lower casing, and integrally coupled with said lower casing, and said upper casing having a containing space;
a rotating body, movably accommodated in said containing space, and having a shaft precisely sheathed onto said pivotal axle; and
a display screen, installed in said rotating body, and accommodated in said containing space;
thereby, said pivotal axle and said shaft are operated together to turn said rotating body to an appropriate position to facilitate viewing said display screen.

2. The sphygmomanometer capable of adjusting a viewing angle of a display screen according to claim 1, wherein said housing is fixed to a cuff, and said cuff is wrapped and fixed onto a users' wrist or arm.

3. The sphygmomanometer capable of adjusting a viewing angle of a display screen according to claim 1, wherein said pivotal axle includes a protruding pillar disposed therein, and said protruding pillar includes a bushing sheathed around said protruding pillar, and a predetermined interval is maintained between said bushing and said protruding pillar.

4. The sphygmomanometer capable of adjusting a viewing angle of a display screen according to claim 1, wherein said rotating body further comprises a chassis, and said shaft is installed on a side of said chassis which faces a side of said lower casing.

5. The sphygmomanometer capable of adjusting a viewing angle of a display screen according to claim 4, wherein said chassis includes a position limit pillar disposed thereon, and protruded from the same direction of said shaft.

6. The sphygmomanometer capable of adjusting a viewing angle of a display screen according to claim 5, wherein said position limit pillar is shorter than said shaft.

7. The sphygmomanometer capable of adjusting a viewing angle of a display screen according to claim 5, wherein said chassis further comprises a positioning element.

8. The sphygmomanometer capable of adjusting a viewing angle of a display screen according to claim 7, wherein said positioning element is an elastic plate.

9. The sphygmomanometer capable of adjusting a viewing angle of a display screen according to claim 7, wherein said positioning element includes a first positioning point protruded from a front end of said positioning element.

10. The sphygmomanometer capable of adjusting a viewing angle of a display screen according to claim 9, wherein said lower casing includes a position limit mechanism, and said position limit mechanism includes a position limit groove.

11. The sphygmomanometer capable of adjusting a viewing angle of a display screen according to claim 10, wherein said position limit mechanism is integrally formed with said lower casing.

12. The sphygmomanometer capable of adjusting a viewing angle of a display screen according to claim 10, wherein said position limit mechanism is a standalone plate of said lower casing.

13. The sphygmomanometer capable of adjusting a viewing angle of a display screen according to claim 10, wherein said position limit groove is substantially a cambered surface.

14. The sphygmomanometer capable of adjusting a viewing angle of a display screen according to claim 10, wherein said position limit groove is provided for passing and sheathing said shaft and said position limit pillar.

15. The sphygmomanometer capable of adjusting a viewing angle of a display screen according to claim 13, wherein said position limit pillar is rotated and limited within a range of said cambered surface of said position limit groove.

16. The sphygmomanometer capable of adjusting a viewing angle of a display screen according to claim 10, wherein said position limit groove includes a plurality of second positioning points disposed around the periphery of said position limit groove.

17. The sphygmomanometer capable of adjusting a viewing angle of a display screen according to claim 16, wherein said second positioning point corresponds with said first positioning point, and said first positioning point is shifted on said second positioning point.

18. The sphygmomanometer capable of adjusting a viewing angle of a display screen according to claim 12, wherein said position limit mechanism includes a circuit control device installed thereon, such that said display screen is electrically coupled with said position limit mechanism through an electric circuit.

19. The sphygmomanometer capable of adjusting a viewing angle of a display screen according to claim 18, wherein said electric circuit is an electric wire or a power cable.

20. The sphygmomanometer capable of adjusting a viewing angle of a display screen according to claim 1, wherein said lower casing includes a at least one screw hole disposed around said lower casing, and said upper casing corresponding to said lower casing includes a screw pillar for passing a screw through said screw hole and secured onto said screw pillar to integrally combine said lower casing and said upper casing.

21. The sphygmomanometer capable of adjusting a viewing angle of a display screen according to claim 1, wherein said lower casing and said upper casing are rectangular objects.
